(19) United States
(12) Patent Application Publication
(10) Pub. No.: US 2024/0335159 A1
(43) Pub. Date: Oct. 10, 2024

(54) METHOD AND SYSTEM FOR ESTIMATING DEMENTIA LEVELS IN AN INDIVIDUAL

(71) Applicant: John Heibel, Baldwin, MI (US)

(72) Inventor: John Heibel, Baldwin, MI (US)

(21) Appl. No.: 18/578,834

(22) PCT Filed: Jul. 14, 2022
(86) PCT No.: PCT/IB2022/056505

§ 371 Date: Jan. 12, 2024

Related U.S. Application Data
(60) Provisional application No. 63/221,551, filed on Jul. 14, 2021.

(51) Int. Cl. A61B 5/00; A61B 5/347

(52) U.S. Cl. CPC A61B 5/4088; A61B 5/347

(57) ABSTRACT

A method and system for estimating dementia levels in a test individual includes using an electrocardiogram to obtain a dataset of ECG measurements captured from the test individual and using a computer based system that is programmed with computer code to convert the dataset of ECG measurements captured from the test individual to a frequency domain. The dataset of ECG measurement in the frequency domain of the test individual is compared with a reference dataset of ECG measurements in the frequency domain of a reference individual in order to estimate a dementia level of the test individual.

### CROSS REFERENCE TO RELATED APPLICATION

The present application claims priority of U.S. provisional application Ser. No. 63/221,551 filed on Jul. 14, 2021, which his hereby incorporated herein by reference in its entirety.

### FIELD AND BACKGROUND OF THE INVENTION

The present disclosure generally relates to a method and system for estimating dementia levels, such as Alzheimer's disease dementia, in an individual based on data collected from the individual's heartbeat.

Alzheimer's disease is the most prevalent type of dementia, making up 60-80% of dementia cases. The disease is primarily categorized by the gradual loss of memory and other cognitive abilities. As a result, it is primarily thought of as a condition that affects the brain. Current ways to diagnose Alzheimer's disease involve direct examination of brain tissue after a patient dies. Doctors currently use brain imaging, evaluation of behavior, psychiatric tests, and other means to diagnose the disease in patients suspected of having Alzheimer's disease, but none are very accurate, and many are costly, time-consuming, and/or not practical.

Consequently, there is a need for an improved method for quickly, reliably, and accurately estimating the state of Alzheimer's disease dementia in an individual. The goal being to help diagnose various stages of Alzheimer's disease in individuals to determine appropriate treatment conditions and regimens.

### SUMMARY OF THE INVENTION

What is provided is an improved method and system for quickly, efficiently, reliably, and accurately estimating the state/amount of Alzheimer's disease dementia in an individual by using data collected from the individual's heartbeat. As a result, data and information is obtained, computed, and analyzed from an electrocardiogram (ECG) is used as the source for dementia calculations.

A method and system for estimating dementia levels in a test individual, according to an aspect of the invention, includes using an electrocardiogram to obtain a dataset of ECG measurements captured from the test individual and using a computer based system that is programmed with computer code to convert the dataset of ECG measurements captured from the test individual to a frequency domain. The dataset of ECG measurement in the frequency domain of the test individual is compared with a reference dataset of ECG measurements in the frequency domain of a reference individual in order to estimate a dementia level of the test individual.

The comparing may be at a plurality of harmonic ranges within the frequency domain. The reference individual may be an individual that is substantially free of dementia.

In an embodiment, a computer system is used to estimate dementia levels of an individual. Software programs, such as MATLAB®, operating on the computer system may be used to conduct various computing, calculating, and analyzing steps in the process of estimating dementia levels in the individual.

In an embodiment, the method for estimating dementia levels of an individual using the computer system includes providing a dataset of electrocardiograms (ECGs) captured from various subjects; identifying the ECG boundaries for the V1-V6 leads; averaging the ECG patterns for the identified V1-V6 leads: computing the frequency content of the ECG signals on one or more of the normalized V1-V6 leads; selecting a reference heartbeat from the ECG data: computing measurements of an individual's averaged V1-V6 lead patterns at various frequency domains; calculating the difference between the amplitudes in the frequency domains and comparing to the waveform data from the reference subjects; and scaling the difference in amplitudes between the frequency domains from 0% to 100% to determine the percentage of dementia and Alzheimer's disease present in a subject.

These and other objects, advantages, purposes and features of this invention will become apparent upon review of the following specification in conjunction with the drawings.

### BRIEF DESCRIPTION OF THE DRAWINGS

The above, as well as other advantages of the present disclosure, will become readily apparent to those skilled in the art from the following detailed description when considered in light of the accompanying drawings in which:

FIG. 1 illustrates a block diagram of an exemplary computer system, in accordance with various embodiment of the present disclosure;

FIG. 2 illustrates an exemplary method for estimating dementia levels of an individual using the computer system illustrated in FIG. 1;

FIG. 3 illustrates an exemplary plot showing difference in amplitudes between the three frequency domains for a first subject;

FIG. 4 illustrates another exemplary plot showing difference in amplitudes between the three frequency domains for a second subject; and

FIG. 5 illustrates yet another exemplary plot showing difference in amplitudes between the three frequency domains for a third subject.

### DETAILED DESCRIPTION OF THE PREFERRED EMBODIMENT

The present invention will now be described with reference to the accompanying figures, wherein the numbered elements in the following written description correspond to like-numbered elements in the figures.

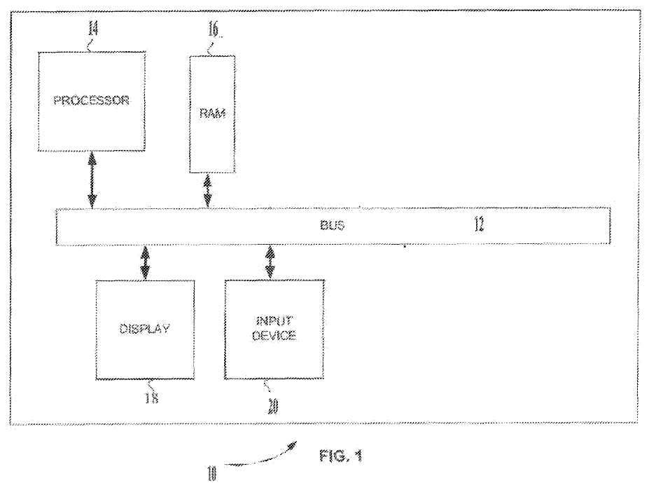

FIG. 1 is a block diagram illustrating an exemplary computer system 10 upon which embodiments of the present disclosure may be implemented, including estimating Alzheimer's disease dementia levels based on data collected from an individual's heartbeat. The computer system 10 includes a bus 12 or other communication mechanism for communicating information, and a processor 14 coupled with the bus 12 for processing information. The computer system 10 includes a memory 16, which may be a random access memory (RAM), read only memory (ROM), and/or other dynamic storage device, coupled to the bus 12 for storing instructions to be executed by the processor 14.

The computer system 10 may be coupled to a display 18 for displaying information to a computer user. An input device 20 is coupled to the bus 12 for communicating information and command selections to the processor 14. The input device 20 may, for example, be a mouse, a trackball, or a cursor for communicating direction information and command selections to the processor 14.

Consistent with certain implementations of the present disclosure, results are provided by the computer system 10 in response to the processor 14 executing one or more sequences of one or more instructions contained in the memory 16. Execution of the sequences of instructions contained in the memory 16 causes the processor 14 to perform methods described herein.

In various embodiments, the computer system 10 may be connected to one or more other computer systems across a network to form a networked system. The network can include a private network or a public network, such as the Internet. The one or more computer systems that store and serve the data may be referred to as servers or the cloud, in a cloud computing scenario. The other computer systems that send and receive data to and from the servers on the cloud may be referred to as client or cloud devices.

The term “computer-readable medium” as used herein refers to any media that participates in providing instructions to the processor 14 for execution. Such a medium may take many forms, including but not limited to, non-volatile media, volatile media, and transmission media. Non-volatile media includes, for example, optical or magnetic disks, such as a storage device 110. Volatile media includes dynamic memory, such as memory 106. Transmission media includes coaxial cables, copper wire, and fiber optics, including the wires that comprise bus 102.

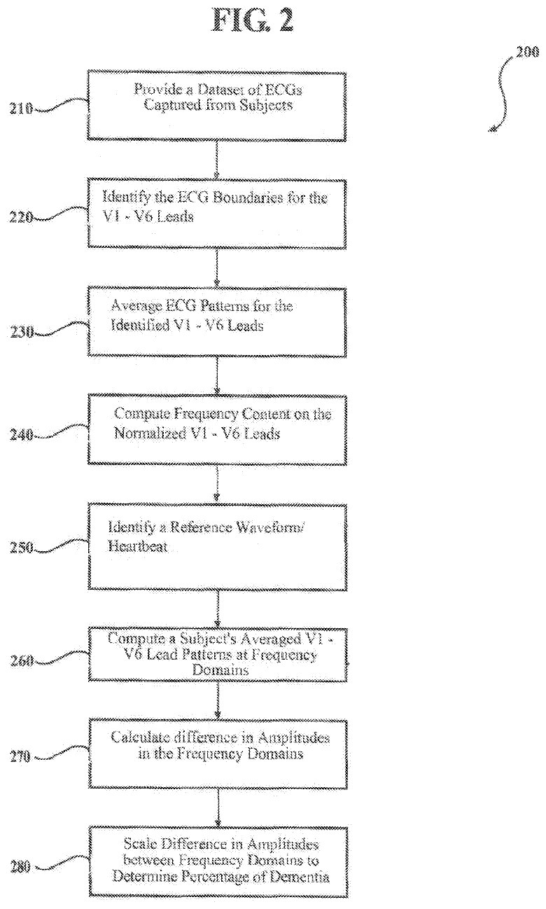

FIG. 2 is an exemplary method 200 for estimating dementia levels of an individual using the computer system 10 illustrated in FIG. 1. The method commences at block 210 by providing a dataset of electrocardiograms (ECGs) captured from various subjects. The ECG is used for detecting information from electrical signals that flow through the different muscle tissues of the heart at it is beating. Electrical signals produced by a beating heart are detected between pairs of electrodes. In an embodiment, the ECGs may be captured by a digital ECG device located at a healthcare professional or a doctor's office, a clinic, or a hospital room. In another embodiment, the ECGs may be captured by a wearable device, such as a smart watch worn by an individual.

The data obtained herein may be from a ECG device having a standard resting 12-lead digital ECG device or a similar measurement device of any number of leads. The digital ECG measurement obtained from an individual may include measured voltages obtained from each lead. For example, the digital ECG measurement extracted from a standard resting digital ECG device for one individual may have 12 sequences representing 12-lead ECG measurements obtained from the individual. A sequence may represent the voltage measurements as a function of time associated with one of the twelve leads: Lead I, Lead II, Lead III, Lead aVR, Lead aVL, Lead aVF, Lead V1, Lead V2, Lead V3, Lead V4, Lead V5, and Lead V6. Leads V1-V6 may correlate to six different chest positions on an individual. Different individuals may have different or similar sequences associated with each lead.

Next, as shown in block 220, the ECG boundaries for the V1-V6 leads are identified using the computer system 10. These boundaries may be computed using one or more software programs operating on the computer system 10, such as MATLAB® or a MATLAB®-based software.

Next, as shown in block 230, the ECG patterns are averaged for the identified V1-V6 leads. The MATLAB® software may be used to measure the amplitude of the identified V1-V6 leads on the ECG. The amplitude is the signal generated by the electrode in response to fluctuations of the electric potential field. As a propagating wave travels closer to an electrode, the ECG amplitude increases, and as it recedes away from the electrode, the amplitude reached a maximum negative value and then diminishes in amplitude as the wave-electrode distance increases. In an embodiment, the V1-V6 leads may be normalized to an amplitude of about 1e4. The value may be scaled up or down provided that it is consistent throughout the entire software program.

Next, as shown in block 240, the frequency content of the ECG signals on one or more of the normalized V1-V6 leads is computed using the computer system 10. In an embodiment, the frequency analysis is performed using a fast Fourier transform (FFT). In other embodiments, other techniques for conducting a frequency analysis or signal processing are performed. The ECG data may be converted into a frequency domain to produce frequency spectra.

A specific, reference heartbeat is then identified from the ECG data, as shown in block 250. The ECG includes waves that correspond to positive or negative deflections from baseline that indicate specific electrical events. In an embodiment, the waves on an ECG include the P wave, Q wave, R wave, S wave, T wave, and U wave. The heartbeat may be selected based on being the “best” heartbeat or waveform by the computer system 10 and/or a human operator. The “best” waveform is determined based on several different features. The “best” waveform/heartbeat that is identified is then referred to Alzheimer's 0 (or 0% Alzheimer's) since it is the least likely to show signs of dementia.

Next, as shown in block 260, measurements of an individual's averaged V1-V6 lead patterns are converted to the frequency domain and computed at various harmonic frequencies to the lowest frequency wave. This may occur through the use of a fast Fornier franform, also known as an FFT, provided in the MATLAB® software via the computer system 10. Since lower frequency harmonics have higher amplitudes than higher frequency harmonic, the low frequency ECG components are the largest in observed amplitude on the ECG. In an embodiment, measurements are taken at three frequency domains: (I) 10-20 harmonics; (II) 20-30 harmonics; and (III) 30-40 harmonics. The data collected from the 10-20 harmonics frequency domain I has been shown to having the dominant relevant in predicting those individuals who either have Alzheimer's disease or who are developing Alzheimer's disease. In other embodiments, a different amount of frequency domains at different harmonics may be measured.

Once the measurements are obtained and converted with the FFT function of MATLAB® software to the harmonics in each of the frequency domain harmonic regions, the difference between the amplitudes in the three frequency domain harmonic regions is calculated and compared to the waveform data from one or more reference subjects that measured 0% Alzheimer's, as shown in block 270. The change in amplitudes in frequency harmonics gets larger in individuals who have or are developing dementia, as compared with a reference subject with no signs of dementia. The difference in amplitudes is based on different frequency domain harmonic ranges. In an embodiment, three frequency domain ranges may be used since the measured amplitudes may be normalized. In another embodiment, the amplitudes measured from one or more of the frequency domain regions may be weighed differently than the amplitudes measured from other frequency domain regions.

As shown in block 280, the difference in amplitudes between the three frequency domains are scaled from 0% to 100% to help determine the percentage of dementia and Alzheimer's disease present in a subject. This may occur through the use of the MATLAB® software via the computer system 10. In an embodiment, the largest difference from all samples of the three frequency domains is classified as 100% Alzheimer's disease. A final score percentage is then calculated to determine an estimate of the percentage of dementia or Alzheimer's disease for a particular subject. An example of this determination is as follows: an estimated score of 0% correlates to no dementia and an estimated score of 100% correlates to Stage 7 of Alzheimer's disease. For example, if the final score percentage is less than 75%, then the subject likely does not need to be treated for Alzheimer's disease. However, an attending physician may prescribe a corrective regimen or medication to prevent the disease from getting worse.

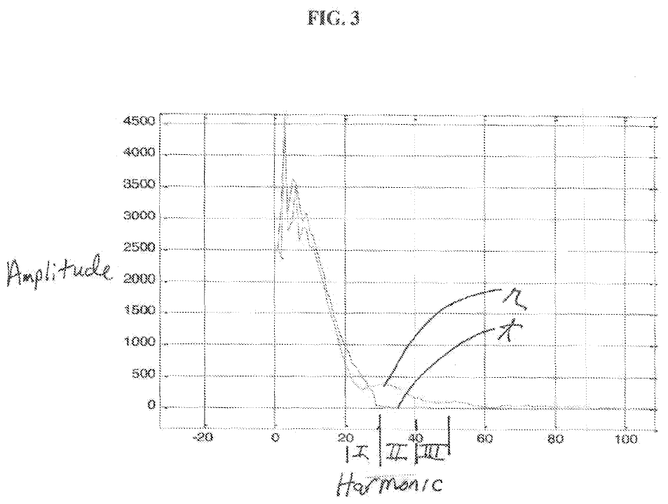

For example, as illustrated in FIG. 3, the differences in amplitude, as expressed in MATLAB® notation, for a first test subject t in each of three (3) frequency domain harmonic ranges (Lo, Mid, Hi) are as follows:
LoDiff=1.7e3 or (1.7×10³);
MidDiff=6.7e2 or (6.7×10²); and
HiDiff=2.7e2 or (2.7×10²).
   The average difference in amplitude between the test subject t and reference individual r for the three frequency domains is 8.8e2 or (8.8×10²) units of amplitude, which corresponds to an estimated dementia/Alzheimer's disease score of 5%.

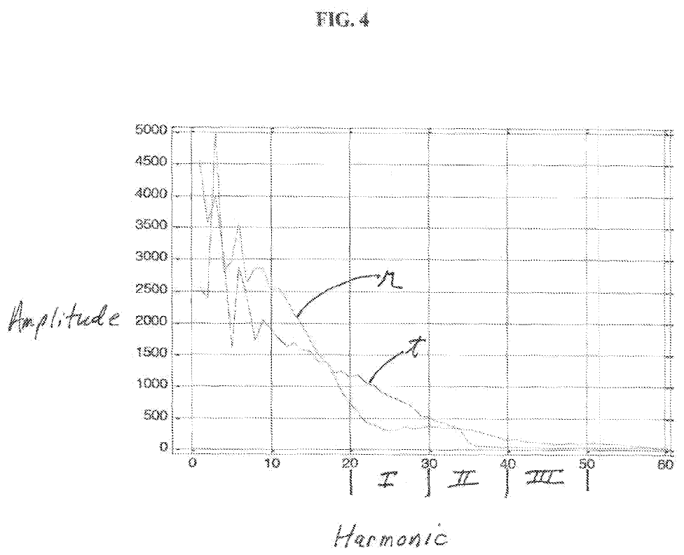

For example, as illustrated in FIG. 4, the differences in amplitude, as expressed in MATLAB® notation, for a second subject in each of three (3) harmonic ranges (Lo, Mid, Hi) are as follows:
LoDiff=1.8e3 or (1.8×10³);
MidDiff=4.5e3 or (4.5×10³); and
HiDiff=8.8e2 or (8.8×10²).
   The average difference in amplitude between the test individual t and reference individual r for the three frequency domain regions is 2.4e3 or (2.4×10³) units of amplitude, which corresponds to an estimated dementia/Alzheimer's disease score of 10%.

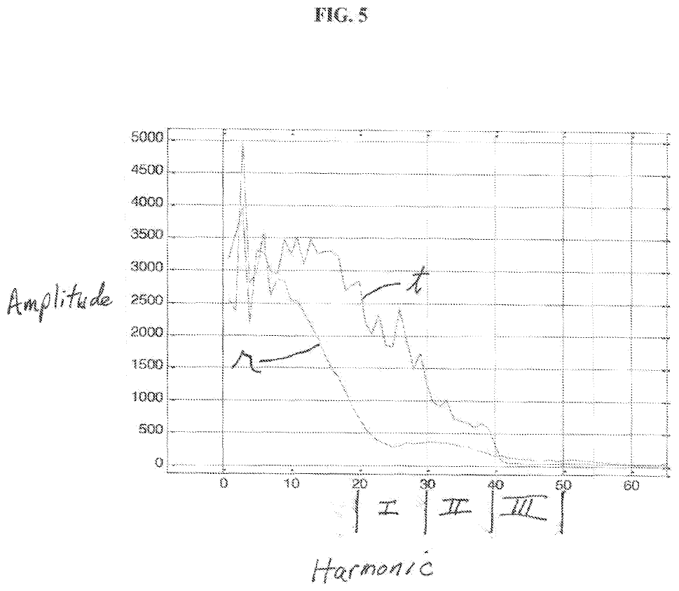

For example, as illustrated in FIG. 5, the differences in amplitude, as expressed in MATLAB® notation, for a third subject t are as follows:
LoDiff=1.5e4 or (1.5×10⁴);
MidDiff=1.5e4 or (1.5×10⁴);
HiDiff=4.4e3 or (4.4×10³).

The average difference between the test individual t and reference individual r in amplitudes for the three frequency domain regions is 1.1 e4 or (1.1×10⁴), which corresponds to an estimated dementia/Alzheimer's disease score of 100%. The third subject was later diagnosed with Alzheimer's disease by a physician.

The method 200 disclosed herein for estimating dementia levels in an individual may occur within about 10 seconds. All of the data computed using the method 200 may then be reported to a healthcare professional, such as a physician, for making a final determination regarding the presence of dementia and/or Alzheimer's disease based on the computed data and estimated score.

Since the heart communicates with the brain when memory is stored by recording the location, the frequencies of the heartbeat help identify where the brain stores memory. The frequencies get larger in amplitude when recording memory during natural placement of that memory. When developing dementia, some frequencies begin migrating since the memory in the brain is not where the heart believes they are. In other words, the heart struggles to identify the location of the frequencies.

It is to be understood that the various embodiments described in this specification and as illustrated in the attached drawings are simply exemplary embodiments illustrating the inventive concepts as defined in the claims. As a result, it is to be understood that the various embodiments described and illustrated may be combined to from the inventive concepts defined in the appended claims.

In accordance with the provisions of the patent statutes, the present disclosure has been described to represent what is considered to represent the preferred embodiments. However, it should be noted that this disclosure can be practiced in other ways than those specifically illustrated and described without departing from the spirit or scope of this disclosure.

1. A method for estimating dementia levels in a test individual, said method comprising:
using an electrocardiogram, obtaining a dataset of ECG measurements captured from the test individual;
using a computer based system that is programmed with computer code converting the dataset of ECG measurements captured from the test individual to a frequency domain; and
using the computer based system, comparing the dataset of ECG measurements in the frequency domain of the test individual with a reference dataset of ECG measurements in the frequency domain of a reference individual in order to estimate a dementia level of the test individual.

2. The method as claimed in claim 1 wherein said comparing being at a plurality of harmonic ranges within the frequency domain, and wherein said comparing comprises scaling a difference in amplitudes in frequency domains to estimate the dementia level based upon the difference in amplitudes.

3. The method as claimed in claim 1, wherein the reference individual is an individual that is substantially free of dementia.

4. A system configured to estimate dementia levels in a test individual, said system comprising:
an electrocardiogram configured to obtain a dataset of ECG measurements captured from the test individual;
a processor configured to process ECG measurements and operable to convert the dataset of ECG measurements captured from the test individual to a frequency domain; and
wherein the processor is operable to compare the dataset of ECG measurements in the frequency domain of the test individual with a reference dataset of ECG measurements in the frequency domain of a reference individual in order to estimate a dementia level of the test individual.

5. The system as claimed in claim 4 wherein the processor is operable to compare the dataset of ECG measurements in the frequency domain of the test individual with a reference dataset of ECG measurements at a plurality of harmonic ranges within the frequency domain.

6. The system as claimed in claim 4, wherein the reference dataset of ECG measurements is from an individual that is substantially free of dementia.

7. The system of claim 4, wherein the processor is operable to compare the dataset of ECG measurements in the frequency domain of the test individual to a reference dataset of ECG measurements in the frequency domain of a reference individual in order to estimate a dementia level of the test individual, wherein the processor is operable to estimate the dementia level by scaling a difference in amplitudes in frequency domains, and wherein the estimate of dementia level is based upon the difference in amplitudes.

8. The system of claim 4, wherein the processor is operable to select the reference dataset of ECG measurements from a plurality of reference datasets of ECG measurements based upon one or more criteria such that the select reference dataset of ECG measurements is least likely to show any signs of dementia.

9. The system of claim 4, wherein the dataset of ECG measurements captured from the test individual comprises a plurality of ECG measurements, and wherein the plurality of ECG measurements is averaged and/or normalized to a common value.

10. The system of claim 4, wherein the processor is operable to convert the dataset of ECG measurements captured from the test individual to a frequency domain by performing a frequency analysis using a fast Fourier transform (FFT).

11. The system of claim 4, wherein the processor is operable to convert the dataset of ECG measurements captured from the test individual to the frequency domain by computing one or more frequency domain ranges, each at a selected different harmonic range.

12. The system of claim 11, wherein the processor is operable to convert the dataset of ECG measurements captured from the test individual to a frequency domain by computing three frequency domains comprising a first frequency domain of 10-20 harmonics, a second frequency domain of 20-30 harmonics, and a third frequency domain of 30-40 harmonics.

13. The method of claim 1 further comprising comparing the dataset of ECG measurements in the frequency domain of the test individual with a reference dataset of ECG measurements in the frequency domain of a reference individual to estimate a dementia level of the test individual.

14. The method of claim 1, wherein the reference dataset of ECG measurements is selected from a plurality of reference datasets of ECG measurements.

15. The method of claim 14, wherein the reference data set of ECG measurements is selected from the plurality of reference datasets of ECG measurements based upon one or more criteria such that the selected reference dataset of ECG measurements is least likely to show any signs of dementia.

16. The method of claim 1, wherein the dataset of ECG measurements captured from the test individual comprises a plurality of ECG measurements, and wherein the plurality of ECG measurements is averaged.

17. The method of claim 1, wherein the dataset of ECG measurements captured from the test individual comprises a plurality of ECG measurements that are each normalized to a common value.

18. The method of claim 1, wherein converting the dataset of ECG measurements captured from the test individual to a frequency domain comprises performing a frequency analysis using a fast Fourier transform (FFT).

19. The method of claim 1, wherein converting the dataset of ECG measurements captured from the test individual to a frequency domain comprises computing one or more frequency domain ranges, each at a selected different harmonic range.

20. The method of claim 19, wherein converting the dataset of ECG measurements captured from the test individual to a frequency domain comprises computing three frequency domains comprising a first frequency domain of 10-20 harmonics, a second frequency domain of 20-30 harmonics, and a third frequency domain of 30-40 harmonics.
